(19) United States
(12) Patent Application Publication
(10) Pub. No.: US 2004/0092781 A1
(43) Pub. Date: May 13, 2004

(54) Preparation of polyhaloalkanes

(76) Inventors: Albrecht Marhold, Leverkusen (DE); Axel Pleschke, Koln (DE)

Correspondence Address: BAYER CHEMICALS CORPORATION, PATENT DEPARTMENT, 100 BAYER ROAD, PITTSBURGH, PA, 15205-9741, US

(21) Appl. No.: 10/703,262

(22) Filed: Nov. 7, 2003

(30) Foreign Application Priority Data
Nov. 11, 2002 (DE) 10252272.3

(51) Int. Cl. C07C 19/08

(52) U.S. Cl. 570/163

(57) ABSTRACT

The present invention relates to a process for preparing polyhaloalkanes and to their use for preparing intermediates for agrochemicals and pharmaceuticals.

### BACKGROUND OF THE INVENTION

1. Field of the Invention

The present invention relates to a process for preparing polyhaloalkanes and to their use for preparing intermediates for agrochemicals and pharmaceuticals.

2. Brief Description of the Prior Art

Polyhaloalkanes, in particular perfluoroalkyl chlorides, bromides and iodides, are valuable starting materials, in particular for preparing polyhaloalkylated aromatics. Polyhaloalkylated aromatics are becoming increasingly important, as lipophilic building blocks in agrochemical or pharmaceutical active ingredients, which ensure good membrane permeability.

The processes for preparing polyhaloalkanes and their disadvantages are described as follows. For example, U.S. Pat. No. 3,770,838 discloses the preparation of polyfluoroalkyl chlorides and bromides by reacting polyfluoroalkenes with potassium fluoride and cyanogen chloride or cyanogen bromide. However, the high toxicity of the cyanogen halides is undesired in industrial application. U.S. Pat. No. 5,057,634 discloses the preparation, inter alia, of 2-chlorohepta-fluoropropane from C₃ building blocks by mixed gas phase chlorination and fluorination with chlorine and hydrogen fluoride. However, low chemoselectivity and the severe conditions for the preparation are disadvantageous. Petrov et al. (J. Fluorine Chem., 2000, 102, 199-204) describe a process for preparing perfluoroalkyl halides by reacting perfluoroalkenes with perfluoroalkyl halides in the presence of aluminium salts. The high requirement for these aluminium salts makes industrial application of the process unviable.

There is, therefore,a need for providing a process which enables the preparation of polyfluoroalkyl chlorides, bromides and iodides in a simple manner, in good yields and under moderate reaction conditions.

### SUMMARY OF THE INVENTION

In accordance with the foregoing, the claimed invention encompasses a process for preparing compounds of the formula (I)

R¹R²CHal¹-CFR³R⁴ (I)

in which

Hal¹ is chlorine, bromine or iodine

R¹, R², R³ and R⁴ are each independently hydrogen, fluorine, chlorine or bromine or C₁-C₁₂-haloalkyl, or in each case two of the R¹, R², R³ and R⁴ radicals together form at least one cyclic radical having a total of 4 to 8 ring atoms,

characterized in that

compounds of the formula (II),

R¹R²CHal¹-CHal²R³R⁴ (II)

in which

Hal¹ and Hal² are each independently as defined above for Hal¹ and R¹, R², R³ and R⁴ are each independently as defined above are converted in the presence of ionic fluoride.

For the purposes of the invention, all radical definitions, parameters and illustrations above and listed hereinbelow, in general or within areas of preference, i.e. the particular areas and areas of preference, may be combined with each other as desired.

### DETAILED DESCRIPTION OF THE INVENTION

Haloalkyl is in each case independently a straight-chain, cyclic, branched or unbranched alkyl radical which is substituted singly, multiply or fully by fluorine and/or chlorine atoms. The term haloalkyl also includes alkyl radicals substituted multiply by fluorine atoms and optionally singly or multiply by chlorine,(referred to hereinbelow as polyfluoroalkyl radicals), and alkyl radicals substituted fully by fluorine atoms, (referred to hereinbelow as perfluoroalkyl radicals).

Examples of the haloakyl is C₁-C₄-haloalkyl selected from the group consisting of trifluoromethyl, 2,2,2-trifluoroethyl, pentafluoroethyl, nonafluorobutyl and difluorochloromethyl, and C₁-C₁₂-haloalkyl is additionally, for example, perfluorocyclohexyl, perfluoro-n-pentyl, perfluoro-n-hexyl and perfluoro-n-dodecyl. Preferably, Hal¹ and Hal² are each independently bromine or chlorine, more preferably identically bromine.

R¹, R², R³ and R⁴ are preferably each independently hydrogen, fluorine, chlorine, C₁-C₄-polyfluoroalkyl or C₁-C₄-perfluoroalkyl, more preferably each independently hydrogen, fluorine, chlorine or C₁-C₄-perfluoroalkyl.

More preferably, in each case three or four radicals from R¹, R², R³ and R⁴ are each fluorine or C₁-C₄-perfluoroalkyl, and no or one radical is hydrogen or chlorine.

Preferred compounds of the formula (I) are 1,2-dibromotetrafluoroethane, 1,2-dibromo-1-chlorotrifluoroethane, 2,3-dibromooctafluorobutane, 2,3-dibromo-2,3-dichlorohexafluorobutane, 2,3-dibromo-2,3-dichlorohexafluorobutane, 2,3-dibromo-1,1,1,3,4,4,4-heptafluorobutane, 2,3-dibromo-2-chloro-1,1,1,4,4,4-hexafluorobutane, 1,2-dibromohexafluoropropane and 1,2-dichlorohexafluoropropane, and particular preference is given to 1,2-dibromohexafluoropropane, 2dibromo-1-chlorotrifluoroethane. Very particular preference is given to 1,2-dibromohexafluoropropane.

The process according to the invention is more preferably suitable for preparing 1-bromopentafluoroethane from 1,2-dibromotetrafluoroethane, 1-bromo-2-chlorotetrafluoroethane and 1-chloro-2-bromotetrafluoroethane from 1,2-dibromo-1-chlorotrifluoroethane, 2-bromononafluorobutane from 2,3-dibromoocta-fluorobutane, 2-bromo-2,3-dichloroheptafluorobutane from 2,3-dibromo-2,3-dichlorohexafluorobutane, 2-bromo-1,1,1,3,3,4,4,4-octafluorobutane and 2-bromo-1,1,1,2,3,4,4,4-octafluorobutane from 2,3-dibromo-1,1,1,3,4,4,4-heptafluorobutane, 2-bromo-2-chloro-1,1,1,3,4,4,4-heptafluorobutane and 2-bromo-3-chloro-1,1,1,3,4,4,4-heptafluorobutane from 2,3-dibromo-2-chloro-1,1,1,4,4,4-hexafluorobutane and 2-bromo-heptafluoropropane from 1,2-dibromohexafluoropropane or 2-chloroheptafluoropropane from 1-bromo-2-chlorohexafluoropropane or 1,2-dichlorohexafluoropropane.

The compounds of the formula (II) are converted in the presence of ionic fluoride.

Ionic fluorides are, for example, quaternary ammonium fluorides or phosphonium fluorides, and also alkali metal fluorides or mixtures of the compounds mentioned.

Examples of ammonium fluorides or phosphonium fluorides are those of the formula (III),

(cation⁺)(F⁻) (III)

in which

(cation⁺) is a cation of the formula (IV)

[pnic(C₁-C₁₂-alkyl)_{q}(C₆-C₁₅-arylalkyl)ᵣ(C₅-C₁₄-aryl)ₛ({(C₂-C₆-alkyl)-O]ᵥ-(C₁-C₆-alkyl)}ₜ)]⁺ (IV)

where

pnic is nitrogen or phosphorus and

(q+r+s+t)=4.

However, preference is given to using alkali metal fluorides or mixtures of alkali metal fluorides, more preferably sodium fluoride, potassium fluoride and caesium fluoride, most preferably potassium fluoride. Preference is given to using potassium fluoride which has a very low water content and a very large surface area.

The molar ratio of ionic fluoride to compound of the formula (II) used may, for example, be from 0.7 to 5, preferably from 0.9 to 2 and more preferably from 1.1 to 1.7. When compounds of the formula (II) which contain no further chlorine atoms are used, the amount of ionic fluoride in principle has no upper limit, but larger amounts are uneconomic.

It has been found that bromine atoms are typically substituted before chlorine atoms and, for the same atom type, the substitution rate reduces drastically in the series primary, secondary, tertiary carbon atom. For example, in the case of the reaction of 1,2-dibromohexafluoropropane, the substitution at the primary carbon atom is observed with a selectivity of greater than 20:1.

Preference is given to carrying out the process in an organic solvent. Suitable organic solvents are, for example: ketones such as acetone, 2-butanone or methyl isobutyl ketone; nitriles such as acetonitrile, propionitrile, benzonitrile, benzyl nitrile or butyronitrile; amides such as N,N-dimethylformamide, N,N-dimethylacetamide, N-methylformanilide, N-methylpyrrolidone, N-methyl-caprolactam or hexamethylphosphoramide; sulphoxides such as dimethyl sulphoxide, sulphones such as tetramethylenesulphone, polyethers such as 1,4-dioxane, ethylene glycol dimethyl ether, ethylene glycol diethyl ether, diethylene glycol dimethyl ether, diethylene glycol diethyl ether, or mixtures of such organic solvents.

The water content of the solvent in the process according to the invention is preferably at a maximum of 1% by weight, preferably at a maximum of 0.2% by weight. Preference is given to achieving such a water content by incipient distillation or drying in a manner known per se. When alkali metal fluorides are used, particular preference is given to drying or incipiently distilling the solvent simultaneously in the presence of the alkali metal fluoride used.

The reaction temperature may, for example, be 60° C. up to the boiling point of the solvent used at the reaction pressure, and up to a maximum of 300° C., preferably 110° C. up to the boiling point of the solvent used at the reaction pressure, and up to a maximum of 200° C.

The reaction pressure may be, for example, 0.8 to 100 bar, and preferably 3 to 25 bar.

The reaction time may, for example, be 10 min 72 hours, and preferably 2 to 12 hours.

Optionally, the reactivity of the ionic fluorides can be modified by additives. Suitable additives are, for example, phase transfer catalysts and/or halex catalysts.

Suitable phase transfer catalysts are, for example, crown ethers such as 18-crown-6,12-crown-4, dibenzo-18-crown-6 or dibenzo-12-crown-4, cryptands such as cryptand[2.2.2] or podands such as polyglycol ethers or those of the formula (V)

(cation⁺)(anion⁻) (V)

in which

(cation⁺) has the definition and areas of preference specified above and

(anion⁻) is the anion of an organic or inorganic acid.

Halex catalysts are, for example, tetrakis(dialkylamino)phosphonium compounds (WO 98/05610) or compounds of the formula (VI)

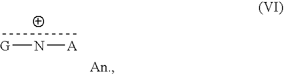

in which

G is a radical of the formulae (VIIa) or (VIIb)

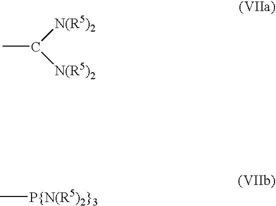

and

H, independently of G, is a radical of the formulae (VIIa), (VIIb), (VIIc) or (VIId)

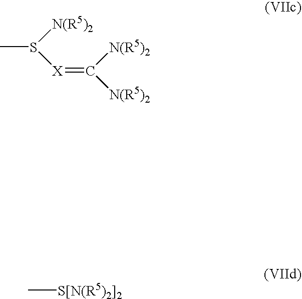

where the radicals

R⁵ are each independently C₁-C₁₂-alkyl, C₂-C₁₀-alkenyl or C₆-C₁₂-aryl,

or where

N(R⁵)₂ as a whole may be a 3- to 5-membered, saturated or unsaturated ring,

or where the radicals of the formula (VIIa) and/or the group

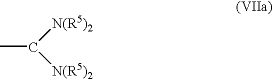

as a whole may be a saturated or unsaturated 4- to 8-membered ring, and

X is nitrogen or phosphorus and

An⊕ is one equivalent of an anion, for example and with preference, chloride, bromide, (CH₃)₃SiF₂⊕, HF₂⊕, H₂F₂⊕, tetrafluoroborate, hexafluorophosphate, carbonate or sulphate.

The compounds of the formula (VI) are obtainable, for example, by reacting compounds of the formula (VIII)

[G—An′]⊕An⊕ (VII)

in which

G and An⊕ are as defined in formula (VI) and

An′ is chlorine or bromine

with compounds of the formula (IX)

HN=G′ (IX)

in which

G′, with regard to the arrangement of the atoms, is as defined for G in formula (VI), but is divalent, and the reaction is effected in the presence of a base.

The compounds of the formula (VI) are described in DE 101 29 057.

The reaction mixture obtained in step a) can be worked up in a manner known per se, typically, for example, by fractional distillation directly from the reaction mixture, optionally under reduced pressure.

The compounds of the formula (II) used as starting products are known from the literature or can be synthesized in a similar manner to the literature. In a preferred embodiment, the compounds of the formula (II) are prepared by

reacting compounds of the formula (X)

R¹—CR²=CR³—R⁴ (X)

with halogen compounds of the formula (XI),

Hal¹-Hal² (XI)

in which

Hal¹ and Hal² are each as defined above under formula (II).

Particularly preferred compounds of the formula (XI) are chlorine and bromine, and more particularly preferred is bromine.

The reaction can be carried out with or without solvent in a manner known per se. Preference is given to carrying it out without solvent. For example, the halogen compound can be initially charged and the compound of the formula (X) added, optionally with cooling.

The reaction temperature may be, for example, −40 to 100° C., preferably 0 to 40° C. and most preferably 15 to 40° C.

The molar ratio of compounds of the formula (XI) to compounds of the formula (III) may, for example, be 0.5 to 3, preferably 0.8 to 1.5 and more preferably 0.9 to 1.2. While larger and smaller amounts are possible, they are uneconomical.

The compounds of the formula (I) obtainable in accordance with the invention are suitable in particular in a process for preparing agrochemicals and pharmaceuticals or intermediates thereof, and are preferably suitable for preparing polyfluoroalkylated aromatic compounds.

A significant advantage of the compounds of the formula (I) obtainable in accordance with the invention is that they can be prepared in a simple manner in high yields from readily available reactants.

### EXAMPLES

### Example 1

### Preparation of 1,2-dibromohexafluoropropane

2357 g of bromine (760 ml, 14.75 mol) were initially charged at room temperature and hexafluoropropene was passed in with constant stirring up to decolorization (19 hours, 2400 g, 16.00 mol). The reaction mixture was purged with nitrogen. In this way, 4710 g of 1,2-dibromohexafluoropropane (95% of theory) were obtained.

### Example 2

### Preparation of 1,2-dibromo-1-chlorotrifluoroethane

155.1 g of bromine (50 ml, 0.97 mol) were initially charged at room temperature and trifluorochloroethylene was passed in with constant stirring up to decolorization (4 hours, 113 g, 0.97 mol). The reaction mixture was purged with nitrogen. In this way, 260 g of 1,2-dibromo-1-chlorotrifluoroethane (96% of theory) were obtained.

### Example 3

### Preparation of 2-bromoheptafluoropropane (a)

An autoclave was initially charged with tetramethylenesulphone (2450 ml) and 352 g of potassium fluoride (6.05 mol), and the mixture was dried by distilling off 250 ml of solvent. 1250 g of 1,2-dibromohexafluoropropane from Example 1 were subsequently added, and the mixture was placed under 3 bar of nitrogen and heated to 125° C., resulting in a pressure of 13.5 bar. Heating was continued at the same temperature for another two hours and then the temperature was increased to 175° C. within three hours. The autoclave was cooled to 0° C. and decompressed, and the product was distilled from the reaction mixture into a cold trap. In this way, 870 g of 2-bromoheptafluoropropane having a purity of 95.8% were obtained (83% of theory).

### Example 4

### Preparation of 2-bromoheptafluoropropane (b)

A 40 I autoclave was initially charged with tetramethylenesulphone (14 300 ml) and 5541 g of calcined potassium fluoride (95.38 mol), and the mixture was dried by distilling off 1500 ml of solvent. 19700 g of 1,2-dibromohexafluoropropane (63.58 mol) from Example 1 were subsequently added at 120° C., and the mixture was placed under nitrogen and heated to 125° C., resulting in a pressure of 10.7 bar. Heating was continued at the same temperature for another five hours. The autoclave was cooled to 30° C. and decompressed, and the product was distilled from the reaction mixture into a cold trap. In this way, 15 168 g of 2-bromoheptafluoropropane having a purity of 99.2% were obtained (95% of theory).

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Process for preparing compounds of the formula (I),
R¹R²CHal¹-CFR³R⁴ (I)
in which
Hal¹ is chlorine, bromine or iodine
R¹, R¹, R³ and R⁴ are each independently hydrogen, fluorine, chlorine or bromine or C₁-C₁₂-haloalkyl, or two of the R¹, R², R³ and R⁴ radicals together form at least one cyclic radical having a total of 4 to 8 ring atoms,
comprising, converting:
compounds of the formula (II),
R¹R²CHal¹-CHal²R³R⁴ (II)
in which
Hal¹ and Hal² are each independently as defined above for Hal¹ and
R¹, R², R³ and R⁴ are each independently as defined above in the presence of ionic fluoride.

2. Process according to claim 1, characterized in that Hal¹ and Hal² are each independently bromine or chlorine.

3. Process according to claim 1, characterized in that R¹ and R⁴ are each independently fluorine, chlorine, C₁-C₄-polyfluoroalkyl or C₁-C₄-perfluoroalkyl.

4. Process according to claim 1, characterized in that R² and R³ are each independently hydrogen, fluorine, chlorine, C₁-C₄polyfluoroalkyl or C₁-C₄-perfluoroalkyl.

5. Process according to claim 1, characterized in that the compounds of the formula (II) used are 1,2-dibromotetrafluoroethane, 1,2dibromo-1-chlorotrifluoroethane, 2,3-dibromooctafluorobutane, 2,3dibromo-2,3-dichlorohexafluorobutane, 2,3-dibromo-2,3dichlorohexafluorobutane, 2,3-dibromo-1,1,1,3,4,4,4-heptafluorobutane, 2,3-dibromo-2-chloro-1,1,1,4,4,4-hexafluorobutane, 1,2-dibromohexafluoropropane or 1,2-dichlorohexafluoropropane.

6. Process according to claim 1, characterized in that 1-bromopentafluoroethane is prepared from 1,2-dibromotetrafluoroethane, 1-bromo-2-chlorotetrafluoroethane and 1-chloro-2-bromotetrafluoroethane from 1,2-dibromo-1-chlorotrifluoroethane, 2bromononafluorobutane from 2,3-dibromooctafluorobutane, 2bromo-2,3-dichloroheptafluorobutane from 2,3-dibromo-2,3dichlorohexafluorobutane, 2-bromo-1,1,1,3,3,4,4,4-octafluorobutane and 2-bromo-1,1,1,2,3,4,4,4-octafluorobutane from 2,3-dibromo-1,1,1,3,4,4,4-heptafluorobutane, 2-bromo-2-chloro-1,1,1,3,4,4,4-heptafluorobutane and 2-bromo-3-chloro-1,1,1,3,4,4,4-heptafluorobutane from 2,3-dibromo-2-chloro-1,1,1,4,4,4-hexafluorobutane and 2-bromoheptafluoropropane from 1,2-dibromohexafluoropropane or 2-chloroheptafluoropropane from 1-bromo-2-chlorohexafluoropropane or 1,2-dichlorohexafluoropropane.

7. Process according to claim 1, characterized in that 2-bromoheptafluoropropane is prepared from 1,2-dibromohexafluoropropane.

8. Process according to claim 1, characterized in that the ionic fluorides used are quaternary ammonium or phosphonium fluorides or alkali metal fluorides or mixtures thereof.

9. Process according to claim 1, characterized in that sodium fluoride, potassium fluoride and/or caesium fluoride are used.

10. Process according to claim 1, characterized in that it is carried out in an organic solvent.

11. Process according to claim 10, characterized in that the maximum water content of the solvent is 1% by weight.

12. Process according to claim 1, characterized in that the reaction temperature is 70° C. up to the boiling point of the solvent used at reaction pressure of 1 to 100 bar.

13. Process according to claim 1, characterized by providing additives effective to modify reactivity of the ionic fluorides.

14. Process according to claim 1, characterized in that the compounds of the formula (II) used as starting materials are prepared by
reacting compounds of the formula (X)
R¹-CR²=CR³—R⁴ (X)
with halogen compounds of the formula (XI),
Hal¹-Hal² (XI)
in which
Hal¹ and Hal² are each as defined above under formula (II).

15. Process according to claim 14, characterized in that the compound of the formula (XI) used is bromine.

16. Process according to claim 14, characterized in that the reaction is carried out without solvent.

17. A process for preparing agrochemicals and pharmaceuticals or intermediates thereof comprising incorporating therein compounds of the formula (I) which had been prepared by a process according to claim 1.

18. The process according to claim 17, characterized in that the compound are polyfluoroalkylated aromatic compounds.
